(19) United States
(12) Patent Application Publication
(10) Pub. No.: US 2025/0367184 A1
(43) Pub. Date: Dec. 4, 2025

(54) CYP11A1 INHIBITOR FOR USE IN THE TREATMENT OF PROSTATE CANCER

(71) Applicant: ORION CORPORATION, Espoo (FI)

(72) Inventors: Tarja IKONEN, Espoo (FI); Annamari VUORELA, Espoo (FI)

(21) Appl. No.: 18/696,317

(22) PCT Filed: Sep. 27, 2022
(86) PCT No.: PCT/FI2022/050646

§ 371 Date: Mar. 27, 2024

(30) Foreign Application Priority Data
Sep. 28, 2021 (FI) 20217146

(51) Int. Cl. A61K 31/454; A61K 31/4166; A61K 31/58; A61P 35/04; C12Q 1/6886

(52) U.S. Cl. CPC A61K 31/454; A61K 31/4166; A61K 31/58; A61P 35/04; C12Q 1/6886; C12Q 2600/106; C12Q 2600/156

(57) ABSTRACT

The invention relates to the use of an activating AR gene alteration as a biomarker for identifying prostate cancer patients who have a higher probability to be responsive to the treatment with a CYP11A1 inhibitor. The invention also relates to a method for treating prostate cancer comprising a) obtaining or having obtained a sample from the patient; b) assaying or having assayed a sample to determine whether the patient has an activating AR gene alteration and c) if the patient has an activating AR gene alteration, treating the patient with a therapeutically effective amount of a CYP11A1 inhibitor.

### TECHNICAL FIELD

The present disclosure relates to a method of treating prostate cancer using a CYP11A1 inhibitor as an active ingredient. The present disclosure provides the use of an activating androgen receptor (AR) gene alteration as a biomarker for identifying patients who have a higher probability to be responsive to the treatment with a CYP11A1 inhibitor.

### BACKGROUND OF THE INVENTION

Prostate cancer is the second most common cancer in men. The majority of prostate cancer deaths are due to the development of metastatic disease that is unresponsive to conventional androgen deprivation therapy (ADT). Androgen deprivation, using either surgical or medical approaches, has been the standard therapy for advanced and metastatic prostate cancer for many decades. It has become clear that the prostate cancer that emerges after androgen deprivation therapy remains dependent upon androgen receptor signalling. The prostate cancer cells that survived or are unresponsive to ADT often gained or exhibit the ability to import low levels of circulating androgens (expressed from adrenal glands), become much more sensitive to these low levels of testosterone, and actually synthesize testosterone within the prostate cancer cell itself. This stage of prostate cancer is termed “castration resistant prostate cancer” or CRPC.

The androgen receptor (AR) is a ligand-inducible steroid hormone receptor that is widely distributed throughout the body and is involved in diverse activities, but its primary and dominant functions are in male sex development and differentiation. It is a member of the nuclear receptor superfamily, with which it shares structural and functional similarity. It contains three principal domains, (i) a hypervariable N-terminal domain which regulates transcriptional activity, (ii) a central highly conserved DNA-binding domain and (iii) a large C-terminal ligand-binding domain (AR-LBD), and a short linker between the DNA-binding domain and the AR-LBD. AR is the chief regulatory intracellular transcription factor for genes involved in the proliferation and differentiation of the prostate.

The AR signalling axis is critical in all stages of prostate cancer. In the CRPC stage, disease is characterized by high AR expression, AR amplification and persistent activation of the AR signalling axis by residual tissue/tumour androgens and by other steroid hormones and intermediates of steroid biosynthesis. Thus, current treatment of CRPC involves androgen receptor signalling inhibitors (ARSi) such as AR antagonists (for example flutamide, nilutamide, bicalutamide, enzalutamide, apalutamide and darolutamide) and androgen synthesis inhibitors (for example CYP17A1 inhibitors including abiraterone acetate).

Although therapies can initially lead to disease regression, eventually a majority of the patients develop a disease that is refractory to currently available therapies. Increased progesterone levels in patients treated with abiraterone acetate has been hypothesized to be one of the resistance mechanisms. Several nonclinical and clinical studies have indicated upregulation of enzymes that catalyse steroid biosynthesis at the late stage of CRPC. Furthermore, it has been addressed that prostate cancer resistance to CYP17A1 inhibition may still remain steroid dependent and responsive to therapies that can further suppress de novo intratumoral steroid synthesis upstream of CYP17A1, such as by CYP11A1 inhibition therapy (Cai, C. et al, Cancer Res., 71 (20), 6503-6513, 2011).

Cytochrome P450 monooxygenase 11A1 (CYP11A1), also called cholesterol side chain cleavage enzyme, is a mitochondrial monooxygenase which catalyses the conversion of cholesterol to pregnenolone, the precursor of all steroid hormones. By inhibiting CYP11A1, the key enzyme of steroid biosynthesis upstream of CYP17A1, the total block of the whole steroid biosynthesis can be achieved. CYP11A1 inhibitors may therefore have a great potential for treating steroid hormone dependent cancers, such as prostate cancer, even in advanced stages of the disease, and especially in those patients who appear to be hormone refractory. Recently, two selective CYP11A1 inhibitors, 2-(isoindolin-2-ylmethyl)-5-((1-(methylsulfonyl)-piperidin-4-yl)methoxy)-4H-pyran-4-one (1A) and 5-((1-(methylsulfonyl)piperidin-4-yl)methoxy)-2-((5-(trifluoromethyl) isoindolin-2-yl)methyl)-4H-pyran-4-one (1B) have entered clinical trials for the treatment of prostate cancer patients.

Activating AR gene alterations such as AR gene amplifications and activating mutations of the ligand binding domain (LBD) of AR are other mechanisms of resistance to anti-androgen treatment. AR gene amplification can lead to overexpression of AR enabling tumour cells to continue AR-dependent growth despite low concentrations of serum androgens. Mutations of AR-LBD can result in functional changes in LBD causing gain-of-function of AR. It has been demonstrated that various point mutations in the AR-LBD can lead to AR activation by weak adrenal androgens, steroidal and non-steroidal ligands, and by mutation driven conversion of AR inhibitors into agonists. For example, F877L point mutation in the AR-LBD has been reported to be associated with enzalutamide resistance in both pre-clinical models and clinical studies. F877L mutation is also detected in a clinically-relevant number of enzalutamide-resistant patients.

There is thus a need for an improved therapy for prostate cancer and a method for identification of patients that are most likely to respond to the therapy.

### SUMMARY OF THE INVENTION

It has been found that CYP11A1 inhibitors, such as 2-(isoindolin-2-yl-methyl)-5-((1-(methylsulfonyl)piperidin-4-yl)methoxy)-4H-pyran-4-one (1A) and 5-((1-(methylsulfonyl)piperidin-4-yl)methoxy)-2-((5-(trifluoromethyl) isoindolin-2-yl)-methyl)-4H-pyran-4-one (1B), are particularly effective in the treatment of prostate cancer patients having an activating AR gene alteration, for example AR gene amplification or an activating AR-LBD mutation. A patient having such activating AR gene alteration was found to have a higher probability to be responsive to the treatment with a CYP11A1 inhibitors, such as compound (1A) or (1B), than a patient who is not having an activating AR gene alteration. Such activating AR gene alteration is therefore also useful as a biomarker for selecting prostate cancer patients who have a higher probability to benefit from the treatment with CYP11A1 inhibitors.

According to one aspect, the present disclosure provides a method for the treatment of prostate cancer in patients having an activating AR gene alteration comprising administration to said patients a therapeutically effective amount of a CYP11A1 inhibitor.

According to another aspect, the present disclosure provides a CYP11A1 inhibitor for use in a method for the treatment of prostate cancer in patients having an activating AR gene alteration.

According to another aspect, the present disclosure provides a method for treating prostate cancer comprising
a) obtaining or having obtained a sample from the patient;
b) assaying or having assayed the sample to determine whether the patient has an activating AR gene alteration; and
c) if the patient has an activating AR gene alteration, treating the patient with a therapeutically effective amount of a CYP11A1 inhibitor.

According to one embodiment, the activating AR gene alteration is an activating AR-LBD mutation. According to another embodiment, the activating AR gene alteration is AR gene amplification.

According to still another aspect, the present disclosure provides a method of selecting a patient suffering from prostate cancer for the treatment with a CYP11A1 inhibitor comprising
a) obtaining or having obtained a sample from the patient;
b) assaying or having assayed the sample to determine whether the patient has an activating AR gene alteration; and
c) if the patient has an activating AR gene alteration, selecting the patient for the treatment with a CYP11A1 inhibitor.

According to one embodiment, the activating AR gene alteration is an activating AR-LBD mutation. According to another embodiment, the activating AR gene alteration is AR gene amplification. In at least one embodiment, the patient selected for treatment with a CYP11A1 inhibitor is administered a therapeutically effective amount of the CYP11A1 inhibitor.

According to another aspect, the present disclosure provides a method for identifying a patient suffering from prostate cancer who is more likely to respond to a treatment comprising a CYP11A1 inhibitor, the method comprising assaying or having assayed a sample obtained from the patient to determine whether the patient has an activating AR gene alteration, wherein such alteration identifies the patient as being more likely to respond to the treatment. In at least one embodiment, the patient selected for treatment with a CYP11A1 inhibitor is administered a therapeutically effective amount of the CYP11A1 inhibitor.

### BRIEF DESCRIPTION OF THE DRAWINGS

FIG. 1 shows the prostate-specific antigen (PSA) change from baseline (%) with or without identified activating AR gene alteration(s) in 37 prostate cancer patients with prior ARSi treatment.

FIG. 2 shows the PSA change from baseline (%) in 16 patients with activating AR-LBD mutation with the identity of mutation(s) in each patient shown.

FIG. 3 shows the PSA change from baseline (%) with or without identified activating AR gene alteration(s) in 25 prostate cancer patients with prior ARSi treatment.

FIG. 4 shows the PSA change from baseline (%) in 15 patients with activating AR-LBD mutation with the identity of mutation(s) in each patient shown.

### DETAILED DESCRIPTION OF THE INVENTION

The present disclosure provides a method for the treatment of prostate cancer in a patient having an activating AR gene alteration, the method comprising administration to said patient a therapeutically effective amount of a CYP11A1 inhibitor. According to one aspect, the CYP11A1 inhibitor is a compound of formula (I) or a pharmaceutically acceptable salt thereof

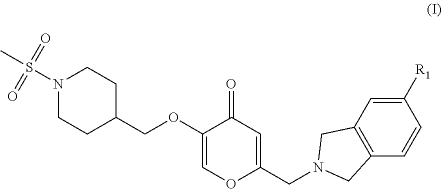

wherein R₁ is hydrogen or —CF₃.

According to another aspect the CYP11A1 inhibitor is 2-(isoindolin-2-yl-methyl)-5-((1-(methylsulfonyl)piperidin-4-yl)methoxy)-4H-pyran-4-one (1A) or a pharmaceutically acceptable salt thereof. According to still another aspect, the CYP11A1 inhibitor is 5-((1-(methylsulfonyl)piperidin-4-yl)methoxy)-2-((5-(tri-fluoromethyl) isoindolin-2-yl)methyl)-4H-pyran-4-one (1B). These compounds have recently entered clinical trials for the treatment of prostate cancer patients.

The results of the clinical studies have shown that a patient having an activating AR gene alteration, has a higher probability to be responsive to the treatment with a CYP11A1 inhibitor than a patient who does not have an activating AR gene alteration.

The term “selective CYP11A1 inhibitor”, as used herein, refers to a compound which selectively binds to CYP11A1 enzyme and suppresses its activity. According to one embodiment, a selective CYP11A1 inhibitor inhibits CYP11A1 at least 100 times, for example at least 500 times, more potently than other drug metabolizing CYP inhibitors including CYP1A2, CYP2B6, CYP2C8, CYP2C9, CYP2C19, CYP2D6, and CYP3A4.

The term “an activating AR gene alteration”, as used herein, refers to alteration of the androgen receptor (AR) which broaden the ligand specificity of the androgen receptor (AR), cause activation of AR by alternative ligands and/or sensitizes AR to low levels of endogenous androgens, for example dihydrotestosterone. Examples of activating AR gene alteration include, but are not limited to, AR gene amplifications and activating AR-LBD mutations.

The term “AR gene amplification” or “AR amplification”, as used herein, refers to formation of extra or multiple copies of the AR gene. Examples of AR gene amplifications include at least 2 copies, at least 3 copies, at least 5 copies, at least 8 copies, at least 10 copies, at least 15 copies and at least 20 copies, of the AR gene.

The term “an activating AR-LBD mutation”, as used herein, refers to a gain-of-function mutation in the ligand binding domain (LBD) of the androgen receptor (AR) which broadens the ligand specificity of the androgen receptor (AR), cause activation of AR by alternative ligands and/or sensitizes AR to low levels of endogenous androgens, for example dihydrotestosterone. The activating AR-LBD mutation may comprise, for example, activating AR-LBD point mutation, activating AR-LBD insertion mutation or activating AR-LBD deletion mutation. In one aspect of the present disclosure, the activating AR-LBD mutation is an activating AR-LBD point mutation.

The term “an activating AR-LBD point mutation”, as used herein, refers to an activating AR-LBD mutation, which is a single amino acid mutation such as a change of a wild-type amino acid to another amino acid in the AR-LBD amino acid sequence.

The human Androgen Receptor amino acid numbering, as used herein, refers to that of UniProt ID: P10275.1 as updated on Mar. 16, 2016. The ligand binding domain (LBD) of the androgen receptor (AR) covers the amino-acid residues 663 to (Wang et al., Acta Cryst., F62, 1067-1071, 2006).

The point mutation nomenclature of AR-LBD, as used herein, follows the standard of depicting wild-type amino acid followed by the amino acid position and the amino acid substitution in mutated variation. For example, the point mutation “L702H” means that amino acid leucine (L) is substituted with amino acid histidine (H) at AR-LBD position 702.

Various activating AR-LBD mutations have been described, for example, in
Shi, X-B. et al., “Functional Analysis of 44 Mutant Androgen Receptors from Human Prostate Cancer”, Cancer Research, 62, 1496-1502, 2002 (AR-LBD point mutations Q671R, I673T, L702H, V716M, K718E, R727L, V731M, A749T, A749V, G751S, V758A, S783N, Q799E, R847G, H875Y, T878A, D891N, A897T, K911R, Q920R);
Lallous, N. et al., “Functional analysis of androgen receptor mutations that confer anti-androgen resistance identified in circulating cell-free DNA from prostate cancer patients”, Genome Biology, 17:10, 1-15, 2016 (AR-LBD point mutations L702H, V716M, V731M, W742C, W742L, H875Y, H875Q, F877L, T878A, T878S, D880E, L8821, S889G, D891H, E894K, M896T, M896V, E898G, T919S,);
Chen, G. et al., “Androgen Receptor Mutants Detected in Recurrent Prostate Cancer Exhibit Diverse Functional Characteristics”, The Prostate, 63, 395-406, 2005 (AR-LBD point mutation E873Q); and
Buchanan, G. et al., “Mutations at the Boundary of the Hinge and Ligand Binding Domain of the Androgen Receptor Confer Increased Transactivation Function”, Molecular Endocrinology, 15 (1), 46-56, 2001 (AR-LBD point mutations Q671R and I673T).

In one aspect of the method of the present disclosure, the patient has one or more of the AR-LBD point mutations selected from a group consisting of Q671R, I673T, L702H, V716M, V716L, K718E, R727L, V731M, W742L, W742C, A749T, A749V, M750I, G751S, V758A, S783N, Q799E, R847G, E873Q, H875Y, H875Q, F877L, T878A, T878S, D880E, L8821, S889G, D891N, D891H, D891Y, E894K, M896T, M896V, A897T, E898G, K911R, T919S and Q920R.

In another aspect of the method of the present disclosure, the patient has one or more of the AR-LBD point mutations selected from a group consisting of L702H, V716M, V716L, W742L, W742C, H875Y, F877L, T878A, T878S, D891Y, M896T and M896V.

In another aspect of the method of the present disclosure, the patient has one or more of the AR-LBD point mutations selected from a group consisting of L702H, V716M, V716L, W742C, H875Y, F877L, T878A, D891Y and M896T.

In another aspect of the method of the present disclosure, the patient to be treated has previously received treatment with androgen receptor signalling inhibitors (ARSi) such as androgen receptor antagonists and CYP17A1 inhibitors, and/or chemotherapeutic agents. Typical androgen receptor antagonist include, but are not limited to, enzalutamide, apalutamide, darolutamide, bicalutamide, flutamide, nilutamide, and pharmaceutically acceptable salts thereof. Typical CYP17A1 inhibitors include, but are not limited to, abiraterone acetate and seviteronel. Typical chemotherapeutic agents include, but are not limited to, docetaxel, paclitaxel and cabazitaxel.

In another aspect of the method of the present disclosure, the patient to be treated has previously received treatment with enzalutamide, apalutamide, darolutamide and/or abiraterone acetate or a pharmaceutically acceptable salt thereof. In another aspect, the patient to be treated has earlier received treatment with enzalutamide and/or abiraterone acetate or a pharmaceutically acceptable salt thereof.

In another aspect of the method of the present disclosure, the patient to be treated is resistant to androgen receptor antagonist therapy or a CYP17A1 inhibitor therapy. In another aspect, the patient to be treated is resistant to treatment with enzalutamide, apalutamide, darolutamide and/or abiraterone acetate or a pharmaceutically acceptable salt thereof. In another aspect, the patient to be treated is resistant to treatment with enzalutamide and/or abiraterone acetate or a pharmaceutically acceptable salt thereof.

The present disclosure further provides a method for treating prostate cancer comprising
a) obtaining or having obtained a sample from the patient;
b) assaying or having assayed the sample to determine whether the patient has an activating AR gene alteration; and
c) if the patient has an activating AR gene alteration, treating the patient with a therapeutically effective amount of a CYP11A1 inhibitor.

According to one embodiment, the activating AR gene alteration is an activating AR-LBD mutation. According to another embodiment, the activating AR gene alteration is AR gene amplification.

The sample may be, for example, a blood sample or a tissue sample. The sample suitably comprises an AR polypeptide or a polynucleotide encoding the AR polypeptide of the patient. In one embodiment, the sample comprises an AR-LBD polypeptide or a polynucleotide encoding the AR-LBD polypeptide of the patient. In one aspect, the method may comprise determining the sequence of the AR (for example AR-LBD) polynucleotide or a portion thereof followed by comparing the sequence of the AR (for example AR-LBD) polynucleotide or polypeptide or a portion thereof of the patient to a wild-type sequence of the AR (for example AR-LBD) polynucleotide or polypeptide or a portion thereof to determine whether the patient has an activating AR gene alteration (for example activating AR-LBD mutation or AR amplification). Alternatively, the sample may be subjected to a suitable gene panel assay targeting the AR region designed to hybrid-capture known AR mutation alterations. A patient having an activating AR gene alteration (for example activating AR-LBD mutation or AR amplification) has been found to have a higher probability to be responsive to the treatment with a CYP11A1 inhibitor than a patient who is not having an activating AR gene alteration (for example activating AR-LBD mutation or AR amplification).

According to one aspect of the present disclosure, the CYP11A1 inhibitor is a selective CYP11A1 inhibitor. According to another aspect of the present disclosure, the CYP11A1 inhibitor is a compound of formula (I) or a pharmaceutically acceptable salt thereof

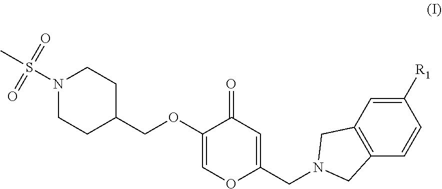

wherein R₁ is hydrogen or —CF₃.

In particular, the compound of formula (I) is 2-(isoindolin-2-ylmethyl)-5-((1-(methylsulfonyl)piperidin-4-yl)methoxy)-4H-pyran-4-one (1A) or 5-((1-(methyl-sulfonyl)piperidin-4-yl)methoxy)-2-((5-(trifluoromethyl) isoindolin-2-yl)methyl)-4H-pyran-4-one (1B), or a pharmaceutically acceptable salt thereof.

The present disclosure further provides a method for selecting a patient suffering from prostate cancer for the treatment with a CYP11A1 inhibitor comprising
a) assaying or having assayed a sample obtained from the patient to determine whether the patient has an activating AR gene alteration; and
b) if the patient has an activating AR gene alteration, selecting the patient for the treatment with a CYP11A1 inhibitor.

According to one embodiment, the activating AR gene alteration is an activating AR-LBD mutation. According to another embodiment, the activating AR gene alteration is AR amplification. In at least one embodiment, the patient selected for treatment with a CYP11A1 inhibitor is administered a therapeutically effective amount of the CYP11A1 inhibitor.

The present disclosure further provides a method for identifying a patient suffering from prostate cancer who is more likely to respond to a treatment comprising a CYP11A1 inhibitor, the method comprising determining whether the patient has an activating AR gene alteration, wherein such alteration identifies the patient as being more likely to respond to the treatment.

According to one embodiment, the activating AR gene alteration is an activating AR-LBD mutation. According to another embodiment, the activating AR gene alteration is AR amplification.

The present disclosure further provides a pharmaceutical composition for use in the treatment of prostate cancer in patients having an activating AR gene alteration, wherein the pharmaceutical composition comprises a CYP11A1 inhibitor as an active ingredient and a pharmaceutically acceptable carrier. A patient having an activating AR gene alteration has a higher probability to be responsive to the treatment with said pharmaceutical composition than a patient who does not have an activating AR gene alteration. According to one embodiment, the activating AR gene alteration is an activating AR-LBD mutation. According to another embodiment, the activating AR gene alteration is AR amplification. In one embodiment, the pharmaceutical composition comprises a compound of formula (I) or a pharmaceutically acceptable salt thereof

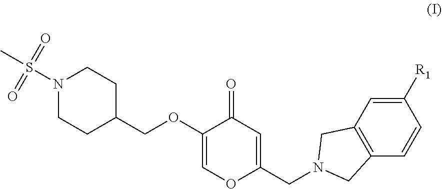

wherein R₁ is hydrogen or —CF₃. In one embodiment, R₁ is hydrogen. In another embodiment, R₁ is —CF₃.

In one aspect, the prostate cancer to be treated is castration resistant prostate cancer (CRPC). In another aspect, the prostate cancer to be treated is metastatic castration resistant prostate cancer (mCRPC). In another aspect, the prostate cancer to be treated is non-metastatic castration resistant prostate cancer (nmCRPC). In still another aspect, the prostate cancer to be treated is castration sensitive prostate cancer (CSPC).

In one aspect, the administration of a CYP11A1 inhibitor, for example a compound of formula (I), to a prostate cancer patient, for example a patient suffering from mCRPC, having an activating AR gene alteration (for example activating AR-LBD mutation or AR amplification) provides an increase in radiographic progression-free survival (rPRS), overall survival and/or a decrease in PSA value. In another aspect, the administration of a CYP11A1 inhibitor, for example a compound of formula (I), to a prostate cancer patient, for example a patient suffering from mCRPC, having an activating AR gene alteration (for example activating AR-LBD mutation or AR amplification) provides an higher increase in radiographic progression-free survival (rPFS), higher increase in overall survival and/or a higher decrease in PSA value compared to a patient not having an activating AR gene alteration (for example activating AR-LBD mutation or AR amplification).

### Sample Preparation and Genomic Profiling

There are a variety of methods that are available for determining if a sample from a patient comprises AR with a particular gene alteration. The methods include, but are not limited to, nucleic acid sequencing (e.g. the methods of DNA sequencing, RNA sequencing, protein sequencing, whole transcriptome sequencing, or other methods known in the art), or using an antibody or nucleic acid specific to the mutation in question. For various references related to sequencing, see for example, Morin et al., Nature 476:298-303 (2011); Kridel et al., Blood 119:1963-1971 (2012); Ren et al., Cell Res. 22:806-821 (2012).

Detection of somatic activating AR gene alterations can be suitably carried out by collecting circulating cell-free DNA (cfDNA) from the plasma of a patient. Whole blood samples are first collected from a patient. The samples are processed to obtain plasma according to a protocol suitable for genomic profiling of cfDNA. The cfDNA is extracted from plasma and somatic activating AR gene alterations originating from prostate tumour cells are detected, for example, by an appropriate hybrid-capture method, suitably using commercially available gene panels targeting to AR region. Examples of suitable methods include, for example, Guardant360 CDx digital next-generation sequencing (NGS) assay available from Guardant Health, Inc. (Odegaard, J. et al., Clin Cancer Res, 2018, 24 (15), 3539-3549) (AR-LBD mutations and AR amplifications), OncoBEAM® digital PCR method available from Sysmex Inostics, Inc. (AR-LBD mutations) as well as FoundationOne Liquid CDx available from Foundation Medicine and Caris Assure™ available from Caris Life Sciences.

Alternatively, obtaining a sample for genomic profiling of the prostate tumour cells of a patient can be carried out by means of conventional tumour tissue biopsy. However, less invasive methods, such as biofluids-based cfDNA methods described above, are preferred.

### In Vitro Functional Testing of AR-LBD Mutations

Whether an observed gene alteration of AR is an “activating AR gene alteration”, as defined herein, can be tested, for example, as follows.

Wild-type human androgen receptor (WT-AR) is encoded on a suitable expression plasmid, for example pcDNA3.1. The AR alterations (for example AR-LBD point mutations) can be generated in the AR cDNA using a site-directed mutagenesis system known in the art. The mutagenic oligonucleotide primers are then designed individually with the desired AR mutation to obtain the AR cDNA of the mutation to be tested. The mutated AR expression plasmid can then be prepared using methods known in the art.

Suitable cells lacking AR expression, for example PC-3 or CV-1 cells, are grown in medium with charcoal-stripped serum (CSS). Cells are co-transfected with wt-AR or altered AR expression plasmids and an AR-driven reporter plasmid like luciferase using transfection reagent. Transiently transfected cells are stimulated with increasing concentrations of tested ligands. The ligands to be tested include endogenous hormonal steroids, as well as antiandrogens and corticosteroids used in the treatment of prostate cancer patients. Endogenous hormonal steroids to be tested include, but are not limited to, testosterone, dihydrotestosterone, progesterone, androstenedione, dehydroepiandrosterone (DHEA), estradiol, cortisol and cortisone. Antiandrogens to be tested include, but are not limited to, bicalutamide, flutamide, hydroxyflutamide, enzalutamide, apalutamide and darolutamide. Corticosteroids to be tested include, but are not limited to, hydrocortisone, prednisone and dexamethasone.

Usually at 24 h after ligand treatment, the medium is aspirated off and the cells are lysed and luciferase activity is measured to determine the AR activation by the ligand. See, for example, Campana, C. et al, Semin Reprod Med. 2015 May; (3): 225-234.

The ligand-induced AR activation of the wild-type AR and altered AR are compared. Higher AR activation of the altered AR by the ligand indicates that the AR gene alteration tested was “an activating AR gene alteration”, as defined herein.

### Methods of Treatment

According to one aspect of the present disclosure, a CYP11A1 inhibitor, for example a compound of formula (I), is administered to a patient having an activating AR gene alteration (for example activating AR-LBD mutation or AR amplification) and suffering from prostate cancer, such as castration resistant prostate cancer (CRPC), for example metastatic castration resistant prostate cancer (mCRPC). A patient having an activating AR gene alteration (for example activating AR-LBD mutation or AR amplification) has a higher probability to be responsive to the treatment with CYP11A1 inhibitor than a patient who is not having an activating AR gene alteration (for example activating AR-LBD mutation or AR amplification). According to one aspect, the patient has previously received an androgen receptor signalling inhibitor (ARSi) such as an androgen receptor antagonist or a CYP17A1 inhibitor, and/or chemotherapy. According to another aspect, the patient is resistant to an androgen receptor signalling inhibitor (ARSi) therapy, for example androgen receptor antagonist therapy and/or CYP17A1 inhibitor therapy.

A CYP11A1 inhibitor may be administered to a patient in therapeutically effective amounts which may range from about 0.1 mg to about 500 mg, or from about 1 mg to about 500 mg, more typically form about 2 mg to about 300 mg, or from about 3 mg to about 150 mg, daily depending on the age, weight, condition of the patient, condition to be treated, administration route and the active ingredient used. When a compound of formula (I) is used for the treatment of prostate cancer, it may be administered to a patient in daily doses which may range from about 0.1 mg to about 300 mg or from about 1 mg to about 300 mg, more typically from about 2 mg to about 150 mg or from about 3 mg to about 100 mg, for example from about 5 mg to about 100 mg, from about 5 mg to about 50 mg or from about 7 mg to about 20 mg.

A CYP11A1 inhibitor is preferably administered with a glucocorticoid and/or a mineralocorticoid and, optionally, with one or more anti-cancer agents. Examples of suitable glucocorticoids include, but are not limited to, hydrocortisone, prednisone, prednisolone, methylprednisolone and dexamethasone. Examples of suitable mineralocorticoids include, but are not limited to, fludrocortisone, deoxycorticosterone, 11-desoxycortisone and deoxycorticosterone acetate. Glucocorticoids may be administered in doses recommended for treating chronic adrenal insufficiency, for example from about 0.2 mg to about 50 mg daily, depending on the glucocorticoid used. Mineralocorticoids may be administered in doses recommended for treating chronic adrenal insufficiency, for example from about 0.01 mg to about 0.5 mg or from about 0.05 mg to about 0.5 mg daily depending on the mineralocorticoid used.

A CYP11A1 inhibitor can be formulated into dosage forms. The compound can be given to a patient as such or in combination with suitable pharmaceutical excipients in the form of tablets, granules, capsules, suppositories, emulsions, suspensions or solutions. Suitable carriers, solvents, gel forming ingredients, dispersion forming ingredients, antioxidants, colours, sweeteners, wetting compounds and other ingredients used to formulate dosage forms may also be used. The compositions containing the active compound can be given enterally or parenterally, the oral route being the preferred way. The contents of the active compound in the composition is from about 0.5 to 100%, for example, from about 0.5 to about 20%, per weight of the total composition.

A CYP11A1 inhibitor, for example a compound of formula (I), may be administered in combination with other anti-cancer treatments useful in the treatment of prostate cancer including, but not limited to, androgen deprivation therapy (ADT), AR antagonists, PARP (poly-ADP ribose polymerase) inhibitors, chemotherapeutic agents (e.g. docetaxel, paclitaxel and cabazitaxel) and radiation therapy.

The invention is further illustrated by the following non-limiting examples.

### EXAMPLES

### Example 1. Clinical Study of Treating Prostate Cancer Patients with a CYP11A1 Inhibitor 2-(Isoindolin-2-ylmethyl)-5-((1-(methylsulfonyl)piperidin-4-yl)-methoxy)-4H-pyran-4-one (1A)

### Methods

Patients with progressive mCRPC were enrolled in the clinical trial to study the effect of a CYP11A1 inhibitor 2-(isoindolin-2-ylmethyl)-5-((1-(methylsulfonyl)-piperidin-4-yl)methoxy)-4H-pyran-4-one (1A). The patients were on ADT therapy and had previously received androgen receptor signalling inhibitor (ARSi) therapy and chemotherapy, or were ineligible for chemotherapy. Five different daily dose levels of compound (1A) with dexamethasone and fludrocortisone were given orally to 27 patients in the dose escalation/de-escalation part. Corticosteroid doses were allowed to be adjusted during the trial based on the subject's clinical condition. The daily doses were 10 mg (5 mg b.i.d), 30 mg (15 mg b.i.d.), 50 mg (25 mg b.i.d), 100 mg (50 mg b.i.d.), and 150 mg (75 mg b.i.d.). In a separate dosing evaluation part, once daily dosing of 25 mg of compound (1A), and two different glucocorticoid replacement therapies, hydrocortisone and prednisone, were evaluated in 14 patients. Subjects were allowed to continue the therapy until disease progression or intolerable toxicity. Anti-tumour activity was determined by measuring change in the PSA (prostate-specific antigen) value in PSA evaluable patient population (n=37, at least 4-week value available). A decrease in the PSA value indicates anti-tumour activity. PSA response in a patient was defined as at least 50% decline from the baseline PSA value.

Existence of activating AR-LBD somatic point mutations and AR gene amplification were analysed in plasma cfDNA samples obtained from the patients using the OncoBEAM® prostate cancer digital PCR assay panel (Sysmex Inostics, Inc.) and Guardant360 CDx (Guardant Health, Inc) assay panel. The assay panels were utilized to test the existence of activating AR-LBD point mutations including L702H, V716M, V716L, W742C, W742L, H875Y, F877L, T878A, T878S, D891Y, M896T and M896V.

### Results

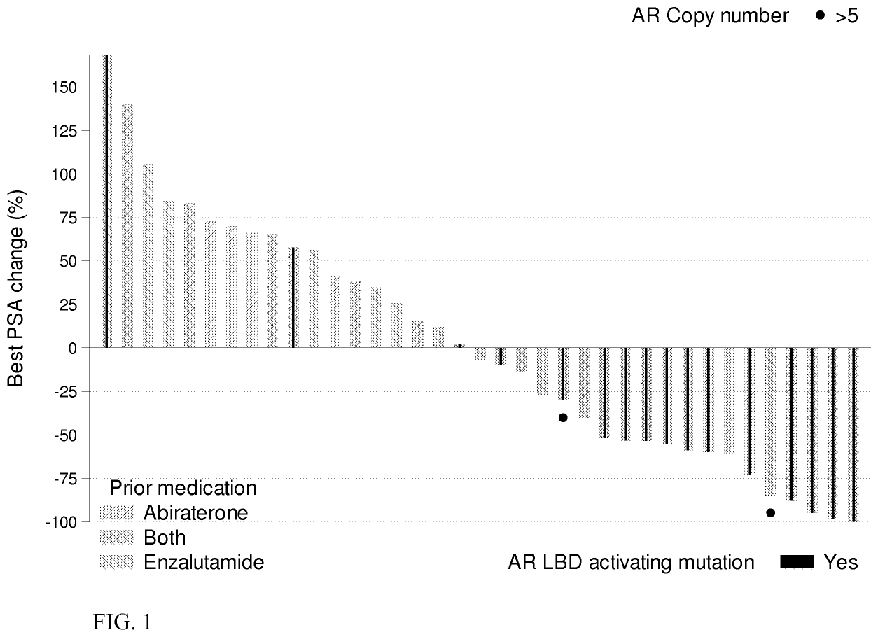

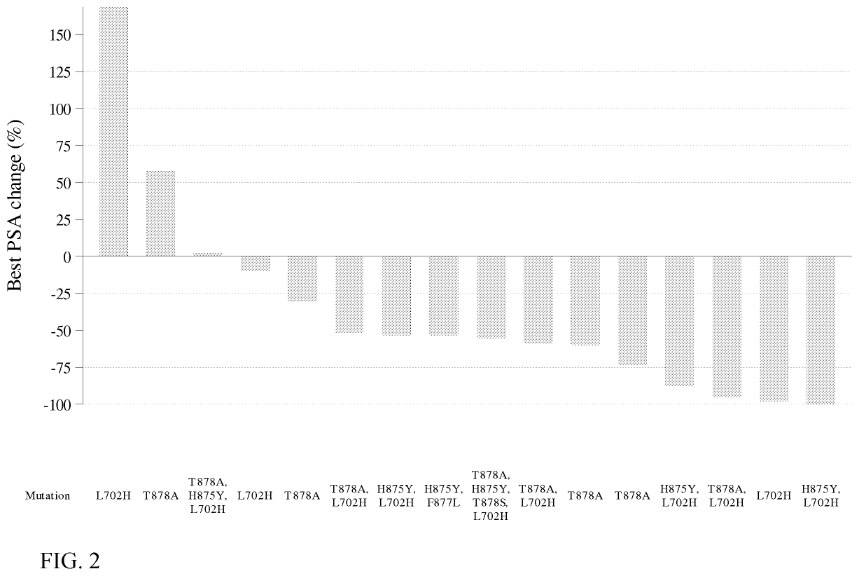

Multiple activating AR-LBD point mutations (from 2 to 4) were detected in 9 subjects, and a single activating AR-LBD point mutation was detected in 7 subjects. AR amplification (>5 copies) was detected in 2 subjects. AR-LBD mutation L702H occurred in 11 subjects, T878A mutation occurred in 9 subjects, H875Y occurred in subjects, F877L occurred in one subject, and T878S in one subject. Anti-tumour activity was observed to be substantially higher in subjects with an activating AR gene alteration (n=17) compared to subjects not having an activating AR gene alteration (n=20). A PSA decline of ≥50% was seen in 70.6% of the subjects (12 out of 17 subjects) with activating AR gene alteration compared to 5.0% of the subjects (1 out of 20) without an activating AR gene alteration. In total, 92.3% of the subjects with a PSA response (decline of ≥50%) were activating AR gene alteration positive (12 out of 13 subjects). PSA responses were also seen in patients who had received androgen receptor signalling inhibitors (ARSi) such as enzalutamide or abiraterone acetate or both. The results are summarized in FIGS. 1 and 2. FIG. 1 shows the PSA change in evaluable 37 patients. The patients having activating AR-LBD mutation (16 patients) are represented by a solid line in the middle of the bar. The patients having AR amplification with >5 copies (2 patients) are represented by a dot below the bar. The previously administered medication is also shown. FIG. 2 shows the PSA change in patients with activating AR-LBD mutation with the identity of mutation(s) in each patient shown.

### Example 2. Clinical Study of Treating Prostate Cancer Patients with a CYP11A1 Inhibitor 5-((1-(methylsulfonyl)piperidin-4-yl)methoxy)-2-((5-(trifluoro-methyl)isoindolin-2-yl)methyl)-4H-pyran-4-one (1B)

### Methods

Patients with progressive mCRPC were enrolled in the clinical trial to study the effect of a CYP11A1 inhibitor 2-5-((1-(methylsulfonyl)piperidin-4-yl)methoxy)-2-((5-(trifluoromethyl) isoindolin-2-yl)methyl)-4/-pyran-4-one (1B). The patients were on ADT therapy and had previously received androgen receptor signalling inhibitor (ARSi) therapy and chemotherapy, or were ineligible for chemotherapy. Three different daily dose levels of compound (1B) with hydrocortisone and fludro-cortisone were given orally to 13 subjects in the dose escalation part. Corticosteroid doses were allowed to be adjusted during the trial based on the subject's clinical condition. The daily doses were 10 mg (10 mg q.d), 15 mg (15 mg q.d) and 20 mg (20 mg q.d). In a separate dosing evaluation part, twice daily dosing of 5 mg and 10 mg of compound (1B), and different glucocorticoid replacement therapy, dexamethasone, and different hydrocortisone dosing regimen were evaluated in 16 patients. Subjects were allowed to continue the therapy until disease progression or intolerable toxicity. Anti-tumour activity was determined by measuring change in the PSA (prostate-specific antigen) value. A decrease in the PSA value indicates anti-tumour activity. PSA response in a patient was defined as at least 50% decline from the baseline PSA value.

Existence of activating AR-LBD somatic point mutations and AR gene amplification were analysed in plasma cfDNA samples obtained from the patients using the OncoBEAM® prostate cancer digital PCR assay panel (Sysmex Inostics, Inc.) and Guardant360 CDx (Guardant Health, Inc) assay panel. The assay panels were utilized to test the existence of activating AR-LBD mutations including L702H, V716M, V716L, W742C, W742L, H875Y, F877L, T878A, T878S, D891Y, M896T and M896V.

### Results

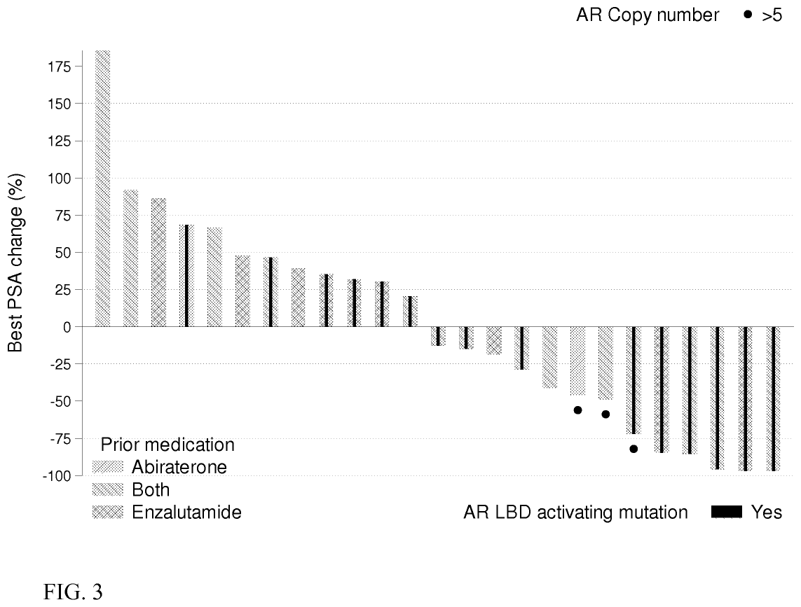

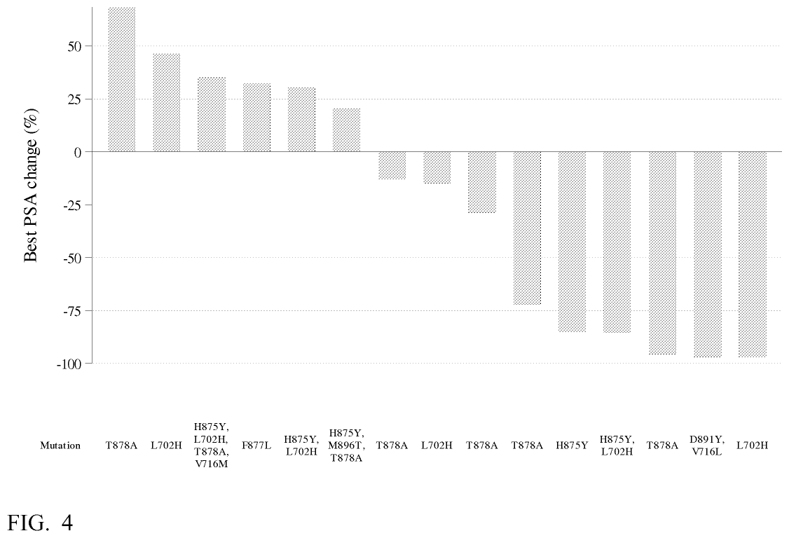

Multiple activating AR-LBD point mutations (from 2 to 4) were detected in 5 subjects, and a single activating AR-LBD point mutation was detected in 10 subjects. AR amplification (>5 copies) was detected in 3 subjects. AR-LBD mutation L702H occurred in 6 subjects, T878A mutation occurred in 7 subjects, H875Y occurred in 5 subjects, and F877L, V716M, M896T, D891Y and V716L each in one subject. Anti-tumour activity was observed to be substantially higher in subjects with activating AR gene alteration (n=17) compared to subjects without an activating AR gene alteration (n=8). A PSA decline of ≥50% was seen in 35.3% of the subjects (6 out of 17 subjects) with activating AR gene alteration compared to 0% of the subjects (0 out of 8) without an activating AR gene alteration. In total, 100% of the subjects with a PSA response (decline of ≥50%) were activating AR gene alteration positive (6 out of 6 subjects). PSA responses were also seen in patients who had received androgen receptor signalling inhibitors (ARSi) such as enzalutamide or abiraterone acetate or both. The results are summarized in FIGS. 3 and 4. FIG. 3 shows the PSA change in evaluable 25 patients. The patients having activating AR-LBD mutation (15 patients) are represented by a solid line in the middle of the bar. The patients having AR amplification with >5 copies (3 patients) are represented by a dot below the bar. The previously administered medication is also shown. FIG. 4 shows the PSA change in patients with activating AR-LBD mutation with the identity of mutation(s) in each patient shown.

1. A method for the treatment of prostate cancer in a patient having an activating androgen receptor (AR) gene alteration comprising administration to said patient a therapeutically effective amount of a CYP11A1 inhibitor.

2. The method according to claim 1, wherein the CYP11A1 inhibitor is a compound of formula (I) or a pharmaceutically acceptable salt thereof 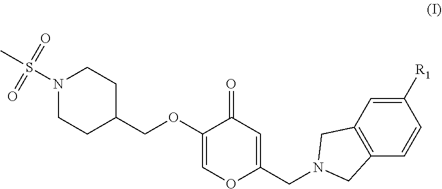
wherein R₁ is hydrogen or —CF₃.

3. The method according to claim 2, wherein R₁ is hydrogen.

4. The method according to claim 3, wherein the patient having an activating AR gene alteration has a higher probability to be responsive to the treatment than the patient who does not have an activating AR gene alteration.

5. The method according to claim 3, wherein the activating AR gene alteration is an AR gene amplification.

6. The method according to claim 3, wherein the activating AR gene alteration is an activating AR-LBD mutation.

7. The method according to claim 6, wherein the activating AR-LBD mutation is an activating AR-LBD point mutation.

8. The method according to claim 7, wherein the patient has one or more of the activating AR-LBD point mutations selected from the group consisting of Q671R, I673T, L702H, V716M, V716L, K718E, R727L, V731M, W742L, W742C, A749T, A749V, M750I, G751S, V758A, S783N, Q799E, R847G, E873Q, H875Y, H875Q, F877L, T878A, T878S, D880E, L882I, S889G, D891N, D891H, D891Y, E894K, M896T, M896V, A897T, E898G, K911R, T919S and Q920R.

9. The method according to claim 8, wherein the patient has one or more of the activating AR-LBD point mutations selected from the group consisting of L702H, V716M, V716L, W742L, W742C, H875Y, F877L, T878A, T878S, D891Y, M896T and M896V.

10. The method according to claim 9, wherein the patient has one or more of the activating AR-LBD point mutations selected from the group consisting of L702H, V716M, V716L, W742C, H875Y, F877L, T878A, D891Y and M896T.

11. The method according to claim 3, wherein the patient has previously received therapy with an androgen receptor antagonist or a CYP17A1 inhibitor.

12. The method according to claim 11, wherein the patient has previously received therapy with enzalutamide or abiraterone acetate or a pharmaceutically acceptable salt thereof.

13. The method according to claim 3, wherein the patient is resistant to androgen receptor antagonist therapy or a CYP17A1 inhibitor therapy.

14. The method according to claim 13, wherein the patient is resistant to treatment with enzalutamide or abiraterone acetate or a pharmaceutically acceptable salt thereof.

15. The method according to claim 3, wherein the prostate cancer to be treated is castration resistant prostate cancer (CRPC).

16. The method according to claim 15, wherein the prostate cancer to be treated is metastatic castration resistant prostate cancer (mCRPC).

17. A method for treating prostate cancer in a patient comprising
a) obtaining or having obtained a sample from the patient;
b) assaying or having assayed the sample to determine whether the patient has an activating AR gene alteration; and
c) if the patient has an activating AR gene alteration, treating the patient with a therapeutically effective amount of a CYP11A1 inhibitor.

18. The method according to claim 17, wherein the CYP11A1 inhibitor is a compound of formula (I) or a pharmaceutically acceptable salt thereof 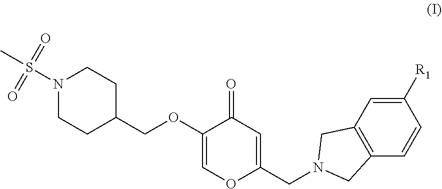
wherein R₁ is hydrogen or —CF₃.

19. The method according to claim 18, wherein R₁ is hydrogen.

20. The method according to claim 19, wherein the patient having an activating AR gene alteration has a higher probability to be responsive to the treatment than a patient who does not have an activating AR gene alteration.

21. The method according to claim 19, wherein the activating AR gene alteration is an AR gene amplification.

22. The method according to claim 19, wherein the activating AR gene alteration is an activating AR-LBD mutation.

23. The method according to claim 19, wherein the sample comprises AR or a portion thereof from the patient.

24. The method according to claim 19, wherein the sample comprises a polynucleotide encoding AR or a portion thereof from the patient.

25. The method according to claim 24, comprising determining the sequence of the AR-LBD polynucleotide or a portion thereof from the patient.

26. The method according to claim 24, comprising subjecting the sample to a gene panel assay targeting the AR-LBD region designed to hybrid-capture known AR-LBD alterations.

27. The method according to claim 19, wherein the prostate cancer to be treated is castration resistant prostate cancer (CRPC).

28. The method according to claim 27, wherein the prostate cancer to be treated is metastatic castration resistant prostate cancer (mCRPC).

29. The method according to claim 19, wherein the prostate cancer to be treated is castration sensitive prostate cancer (CSPC).

30. The method according to claim 29, wherein the prostate cancer to be treated is metastatic castration sensitive prostate cancer (mCSPC).

31. A method of selecting a patient suffering from prostate cancer for the treatment with a CYP11A1 inhibitor comprising
a) assaying or having assayed the sample to determine whether the patient has an activating AR gene alteration; and
b) if the patient has an activating AR-LBD gene alteration, selecting the patient for the treatment with a CYP11A1 inhibitor.

32. The method according to claim 31, wherein the CYP11A1 inhibitor is a compound of formula (I) or a pharmaceutically acceptable salt thereof 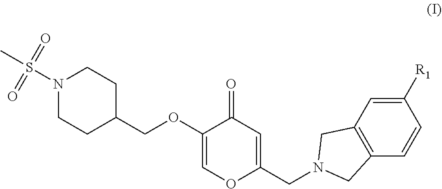
wherein R₁ is hydrogen or —CF₃.

33. The method according to claim 32, wherein R₁ is hydrogen.

34. The method according to claim 31, wherein the patient having an activating AR gene alteration has a higher probability to be responsive to the treatment than a patient who does not have an activating AR gene alteration.

35. The method according to claim 31, wherein the patient suffers from castration resistant prostate cancer (CRPC).

36. The method according to claim 35, wherein the patient suffers from metastatic castration resistant prostate cancer (mCRPC).

37. The method according to claim 31, wherein the patient suffers from castration sensitive prostate cancer (CSPC).

38. The method according to claim 37, wherein the patient suffers from metastatic castration sensitive prostate cancer (mCSPC).

39. A method for identifying a patient suffering from prostate cancer who is more likely to respond to a treatment comprising a CYP11A1 inhibitor, the method comprising assaying or having assayed a sample obtained from the patient to determine whether the patient has an activating AR gene alteration, wherein such alteration identifies the patient as being more likely to respond to the treatment.

40. The method according to claim 39, wherein the CYP11A1 inhibitor is a compound of formula (I) or a pharmaceutically acceptable salt thereof 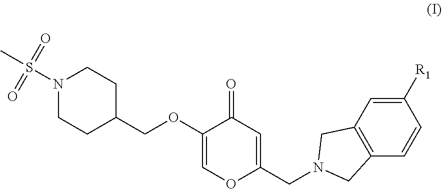
wherein R₁ is hydrogen or —CF₃.

41. The method according to claim 40, wherein R₁ is hydrogen.

42-45. (canceled)

46. The method according to claim 2, wherein the CYP11A1 inhibitor is a pharmaceutically acceptable salt of the compound of formula (I) 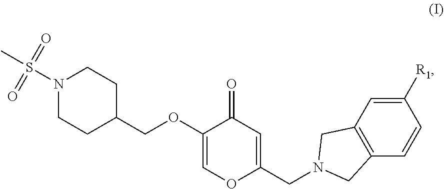
wherein R₁ is hydrogen.

47. The method according to claim 2, wherein the CYP11A1 inhibitor is a compound of formula (I) 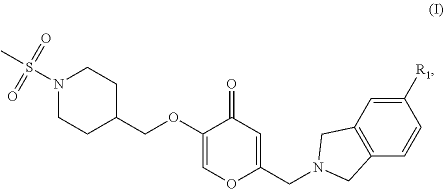
wherein R₁ is hydrogen.

48. The method according to claim 2, wherein the CYP11A1 inhibitor is a pharmaceutically acceptable salt of the compound of formula (I) 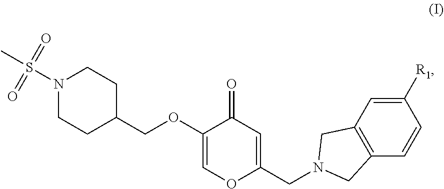
wherein R₁ is —CF₃.

49. The method according to claim 2, wherein the CYP11A1 inhibitor is a compound of formula (I) 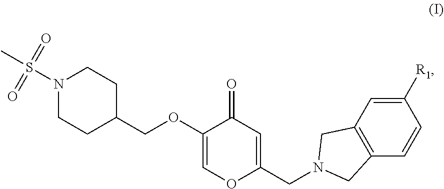
wherein R₁ is —CF₃.

50. The method according to claim 18, wherein the CYP11A1 inhibitor is a compound of formula (I) 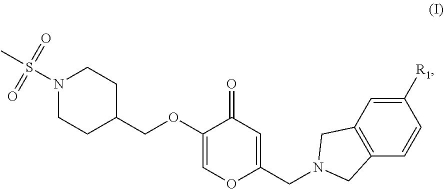
wherein R₁ is —CF₃.

51. The method according to claim 18, wherein the CYP11A1 inhibitor is a pharmaceutically acceptable salt of the compound of formula (I) 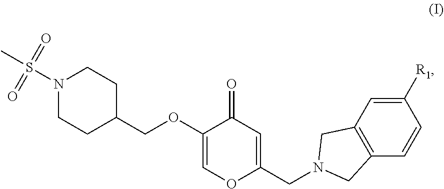
wherein R₁ is —CF₃.

52. The method according to claim 18, wherein the CYP11A1 inhibitor is a pharmaceutically acceptable salt of the compound of formula (I) 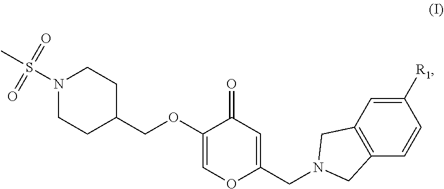
wherein R₁ is hydrogen.

53. The method according to claim 18, wherein the CYP11A1 inhibitor is a compound of formula (I) 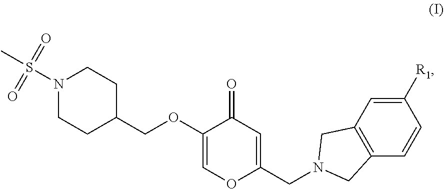
wherein R₁ is hydrogen.
